(19) United States
(12) Patent Application Publication
(10) Pub. No.: US 2006/0235277 A1
(43) Pub. Date: Oct. 19, 2006

(54) Endoscope system

(75) Inventors: Kazunobu Ohkubo, Kanagawa-ken (JP); Masami Hatori, Kanagawa-ken (JP); Shinji Takeuchi, Saitama-shi (JP); Tadashi Ando, Saitama-shi (JP)

Correspondence Address: SUGHRUE MION, PLLC, 2100 PENNSYLVANIA AVENUE, N.W., SUITE 800, WASHINGTON, DC, 20037, US

(73) Assignees: FUJI PHOTO FILM CO., LTD.; FUJINON CORPORATION

(21) Appl. No.: 11/406,342

(22) Filed: Apr. 19, 2006

(30) Foreign Application Priority Data
Apr. 19, 2005 (JP) 121258/2005

(51) Int. Cl. A61B 1/06

(52) U.S. Cl. 600/179; 600/182

(57) ABSTRACT

An endoscope system has an insertion portion and a control portion connected to a base end portion of the insertion portion. An illumination system includes a light guide having a front end positioned near the front end of the insertion portion and a rear end positioned rearward of the base end of the insertion portion, phosphors disposed in the light guide toward the front end thereof and an illumination light source which is positioned toward the rear end of the light guide and emits stimulating light exciting the phosphors.

### BACKGROUND OF THE INVENTION

1. Field of the Invention

This invention relates to an endoscope system having an insertion portion and a control portion connected to a base end portion of the insertion portion, and more particularly to an endoscope system provided with an illumination means for radiating illumination light from the front end of the insertion portion.

2. Description of the Related Art

There has been known, as an illumination means for an endoscope system for medical or industrial use, an illumination means comprising a light source such as a halogen lamp or a xenon lamp, and a light guide which comprises a fiber bundle and transfers illumination light to the front end of the insertion portion. However, such an illumination means involves a problem that it is cumbersome and requires a large power consumption, e.g., hundreds of watts is required for the light source. Accordingly, there has been developed an endoscope having an illumination means employing an LED or LD which is a semiconductor element small in size and the power consumption, e.g., only power as small as several watts is required to drive such a semiconductor light emitting element.

White light is generally necessary as an illumination means for an endoscope. In order to generate white light with a semiconductor light emitting element, a combination of semiconductor light emitting elements emitting light in red, emitting light in green and emitting light in blue is often used. However, when the three color semiconductor light emitting elements are used in combination, there is a problem that unevenness in color is generated. In order to overcome this problem, there is proposed in Japanese Unexamined Patent Publication No. 10(1998)-216085, an illumination means for an endoscope which is provided with a semiconductor light emitting element emitting light having a predetermined wavelength and phosphors excited by the light emitted from the semiconductor light emitting element to emit white fluorescence light at a front end of the insertion portion, and in which the white fluorescence light emitted from the phosphors is used as illumination light.

However, since being a heating body, the semiconductor light emitting element can increase the temperature of the surroundings when it is used long. Especially, as the insertion portion of the endoscope has been reduced in its diameter, the heat capacity of the insertion portion tends to reduce, and when a semiconductor light emitting element is positioned in the insertion portion, there is a fear that the temperature of the surroundings of the semiconductor light emitting element is elevated to an undesirable temperature.

### SUMMARY OF THE INVENTION

In view of the foregoing observations and description, the primary object of the present invention is to provide an endoscope system having an illumination means which can emit illumination light without unevenness in color without increasing the temperature in the insertion portion.

In accordance with the present invention, there is provided an endoscope system having an insertion portion and a control portion connected to a base end portion of the insertion portion, wherein the improvement comprises

an illumination means comprising a light guide having a front end which is positioned near the front end of the insertion portion and a rear end which is positioned rearward of the base end of the insertion portion, phosphors disposed in the light guide toward the front end thereof and an illumination light source which is positioned toward the rear end of the light guide and emits stimulating light exciting the phosphors.

The illumination light source may be positioned in the control portion.

As the illumination light source, a semiconductor light emitting element such as a light emitting diode and a semiconductor laser may be preferably used, and a semiconductor laser is more preferable.

The light guide may comprise a single optical fiber or a plurality of optical fibers bundled together.

The stimulating light may be not shorter than 350 nm and not longer than 500 nm in wavelength.

So long as the illumination means emits white light, the white light may be fluorescence light emitted from the phosphors in response to projection of the stimulating light or may be formed by the stimulating light and fluorescence light emitted from the phosphors in response to projection of the stimulating light.

Since, in the endoscope system having an insertion portion and a control portion connected to a base end portion of the insertion portion, the endoscope system of this invention is characterized by having an illumination means comprising a light guide having a front end which is positioned near the front end of the insertion portion and a rear end which is positioned rearward of the base end of the insertion portion, phosphors disposed in the light guide toward the front end thereof and an illumination light source which is positioned toward the rear end of the light guide and emits stimulating light exciting the phosphors, it is not necessary to position the light source in the insertion portion and accordingly, the endoscope system of this invention can emit illumination light without unevenness in color without increasing the temperature in the insertion portion.

Even when an image pick-up element such as a CCD is employed, that dark noise and the like in the image pick-up element is increased due to increase in the temperature to deteriorate in the S/N of the obtained image can be avoided.

When the illumination light source is positioned in the control portion, it is not necessary to establish connection of the light guide with the illumination light source by complicatedly running cable, whereby the overall size of the endoscope system can be reduced and convenience to handle of the endoscope system can be improved.

When a semiconductor light emitting element is employed as the illumination light source, the overall size of the endoscope system can be reduced and the endoscope system can be produced at low cost. Further, since the semiconductor light emitting element is smaller in power consumption as compared with a halogen lamp or a xenon lamp, the overall size of the power source for the light source can also be reduced. Further, since the semiconductor emitting element is better in light collection as compared with the above mentioned lamps, reduction of the diameter of the light guide is facilitated. The semiconductor light emitting element is easy to position in the control portion of the endoscope system.

When a semiconductor laser is employed as the light source, the light coupling efficiency between the semiconductor laser and the light guide is increased since a laser beam is easy to converge on a fine point, and the number of the semiconductor light emitting element can be reduced or the power consumption can be reduced. Further, when the light coupling efficiency is high, the amount of light to be projected onto the site other than the light inlet end of the light guide or to be scatter at the junction becomes less and accordingly, it becomes possible to suppress heat generation near the junction. Further, a light guide comprising a single optical fiber can be easily employed and, at the same time, the single optical fiber can be reduced in its diameter.

When the stimulating light is not shorter than 350 nm and not longer than 500 nm in wavelength, fluorescence light in a visible region can be efficiently emitted from the phosphors.

### BRIEF DESCRIPTION OF THE DRAWINGS

FIG. 1 is a view briefly showing structure of an endoscope system in accordance with a first embodiment of the present invention,

FIG. 2 is a view briefly showing structure of an endoscope system in accordance with a second embodiment of the present invention,

FIG. 3 is a view briefly showing structure of an endoscope system in accordance with a third embodiment of the present invention, and

FIG. 4 is a view briefly showing structure of an endoscope system in accordance with a fourth embodiment of the present invention.

### DESCRIPTION OF THE PREFERRED EMBODIMENTS

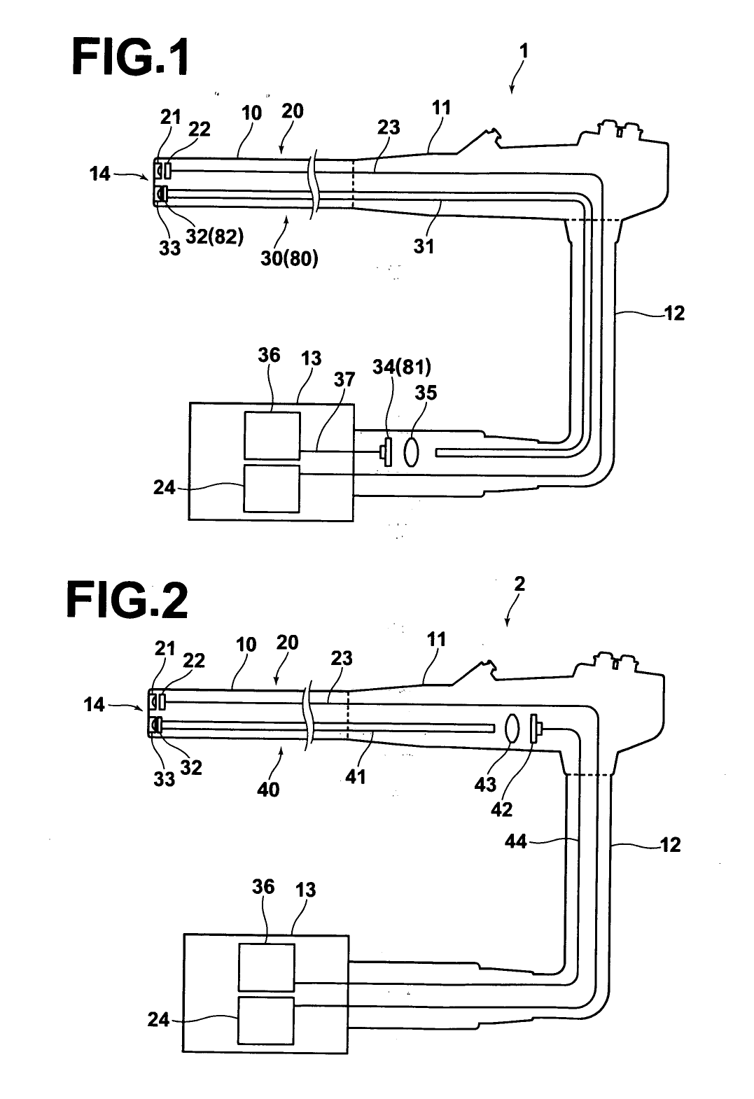

Fluorescence endoscope systems in accordance with embodiments of the present invention will be described with reference to the drawings, hereinbelow. In FIG. 1, a fluorescence system 1 in accordance with a first embodiment of the present invention comprises an insertion portion 10 which is inserted into a body cavity or the like, a control portion 11 which is connected to a base end portion of the insertion portion 10 to control a bend or the like thereof, a universal cable 12 connected to the control portion 11 and a processor 13 removably connected to the universal cable 12. The processor 13 is connected to a monitor not shown. Further, the processor 13 effects a general control of the endoscope system including drive of a CCD 22 and a semiconductor laser 34 to be described later.

The insertion portion 10 comprises a soft portion and an angle portion which is connected to the soft portion and directs a front end portion 14 of the insertion portion 10 in a desired direction.

The endoscope system 1 is further provided with an observing means 20 and an illumination means 30. The observing means 20 and the illumination means 30 are accommodated in the insertion portion 10, control portion 11, universal cable 12 and the processor 13.

The observing means 20 comprises an objective lens 21 provided in the front end portion 14 of the insertion portion 10, a CCD 22 provided in an imaging position of the objective lens 21, a CCD cable 23 which is connected to the CCD 22 and extends to the processor 13 by way of the insertion portion 10, control portion 11 and the universal cable 12, and a signal processing portion 24 which is connected to the CCD cable 23 and is provided in the processor 13. The CCD 22 is provided with an on-chip RGB color filter (not shown).

The illumination means 30 comprises a light guide 31 which is positioned in the front end portion 14 of the insertion portion 10 at its front end and in the universal cable 12 at its rear end, phosphors 32 positioned on the side of the light guide 31 near the front end thereof or in the front end portion 14 of the insertion portion 10, an illumination lens 33 provided in front of the phosphors 32, a semiconductor laser 34 which is positioned in the universal cable 12 on the side of the light guide 31 near the rear end thereof and emits a laser beam which excites the phosphors 32, a collective lens 35 which collects the laser beam emitted from the semiconductor laser 34 on the light inlet end face of the light guide 31, a power source 36 which is provided in the processor 13 and supplies power to the semiconductor laser 34, and a power supply cable 37 connecting the power source 36 and the semiconductor laser 34. The semiconductor laser 34 is provided in the universal cable 12 near the junction with the processor 13, e.g., in a connector. The light guide 31 may comprise a single optical fiber or a plurality of optical fibers bundled together. When the light guide 31 comprises a single optical fiber, the light guide 31 can be easily as small as not larger that 1 mm in its diameter. In this case, the light transmitted through the optical fiber may be single or multiple in its transverse mode.

The semiconductor laser 34 is a GaN series semiconductor laser and emits a laser beam (stimulating light) having a wavelength of near 405 nm (395 to 425 nm).

The phosphors 32 are a mixture of fluorescence material Y₂O₂S:Eu³⁺ which emits red fluorescence upon projection of the stimulating light of 405 nm, fluorescence material ZnS:Cu,Al which emits green fluorescence upon projection of the stimulating light of 405 nm, and fluorescence material (Sr,Ca,Ba,Mg)₁₀(pO₄)₆Cl₂:Eu⁺² which emits blue fluorescence upon projection of the stimulating light of 405 nm, emit white fluorescence light, a mixture of red fluorescence light green fluorescence light and blue fluorescence light, upon projection of the stimulating light.

The fluorescence materials employed in the phosphors 32 need not be limited to those described above but may be suitably selected from various fluorescence materials so that white light is emitted from the phosphors 32 upon projection of the stimulating light. For example, at least one kind of red-fluorescence-light-emitting fluorescence material, at least one kind of green-fluorescence-light-emitting fluorescence material and at least one kind of blue-fluorescence-light-emitting fluorescence material may be selected from the fluorescence materials listed in the following table 1.

TABLE 1

| color | phosphor | λmax |
|---|---|---|
| red | Y₂O₂S:Eu³⁺ | 627 nm |
| | La₂O₂S:Eu³⁺ | 624 nm |
| | Ba₃MgSi₂O₈:Eu²⁺, Mn²⁺ | 620 nm |
| | Sr₃MgSi₂O₈:Eu²⁺, Mn²⁺ | 683 nm |
| | Ca₃MgSi₂O₈:Eu²⁺, Mn²⁺ | 705 nm |
| | LiEuW₂O₈ | 614 nm |
| | Ca₃SiN₂:Eu²⁺ | 630 nm |
| | CaAlSiN₃:Eu²⁺ | 650 nm |
| green | ZnS:Cu, Al | 530 nm |
| | BaMg₂Al₁₆O₂₇:Eu²⁺, Mn²⁺ | 513 nm |
| | Ca₃Sc₂Si₃O₁₂:Ce³⁺ | 503 nm |
| | SrGa₂S₄:Eu²⁺ | 532 nm |
| | CaSi₉Al₃ON₁₅:Yb³⁺ | 549 nm |
| blue | (Sr,Ca,Ba,Mg)₁₀(pO₄)₆Cl₂:Eu⁺² | 448 nm |
| | BaMgAl₁₀O₁₇:Eu²⁺ | 450 nm |
| | CaSi₉Al₃ON₁₅:Ce³⁺ | 477 nm |

Operation of the endoscope system 1 in accordance with the first embodiment of the present invention described above will be described, hereinbelow. A power source 36 is first turned on in response to operation of a manual switch not shown, and a laser beam of 405 nm is emitted from the semiconductor laser 34. The laser beam is collected by the collective lens 35 and enters the light guide 31. The laser beam propagated in the light guide 31 is emitted from the front end thereof which is positioned in the front end portion 14 of the insertion portion 10. The phosphors 32 are excited upon projection of the laser beam to emits white fluorescence light, a mixture of red, green, and blue fluorescence light. The white fluorescence light is projected onto an object part by way of the illumination lens 33 as illumination light.

An image of the object part illuminated with the illumination light is formed on the image pick-up face of the CCD 22 by the objective lens 21. The CCD 22 takes the image of the object part and outputs an image signal representing the image of the object part to the CCD cable 23. The image signal is input into the signal processing portion 24 after propagating through the CCD cable 23. In the signal processing portion 24, predetermined image processing is carried out on the input image signal to generate an image signal for an image display and the image signal for an image display is output to a monitor not shown. The observer observes an image of the object part displayed on the monitor.

As can be understood from the description above, since the endoscope system 1 of the first embodiment is provided with an illumination means 30 comprising a light guide 31 having a front end which is positioned near the front end portion 14 of the insertion portion 10 and a rear end which is positioned in the universal cable 12, phosphors 32 disposed in the light guide 31 toward the front end thereof and a semiconductor laser 34 which is positioned in the universal cable 12 toward the rear end of the light guide 31 and emits a laser beam exciting the phosphors 32, it is not necessary to position the light source in the insertion portion 10 and the endoscope system of this embodiment can emit illumination light without unevenness in color without increasing the temperature in the insertion portion.

Further, since white light can be formed by the use of the phosphors 32, the light source itself need not emit white light or RGB light and may be simply a semiconductor laser which emits a laser beam of 405 nm. Accordingly, the light source can be small in size and can produced at low cost. Further, heat generation of the light source can be suppressed. Further, since the semiconductor laser 34 is small in power consumption, the power source for the light source also can be small in size. Further, since being small in size, the semiconductor laser 34 can be readily accommodated in the universal cable 12.

Further, since a laser beam is easy to converge on a fine point by a lens, a major part of a laser beam emitted from the semiconductor laser 34 enters the light guide 31. Accordingly, even an output of about 200 mW to 400 mW of the semiconductor laser 34 can produce an output not lower than 8 lumen at the light outlet end of the light guide 31. Though a plurality of semiconductor lasers are generally used as the light source when the amount of light at the light outlet end of the light guide 31 is insufficient, the number of the semiconductor lasers to be used as the light source can be reduced even in such a case so long as light coupling efficiency between the semiconductor laser and the light guide is high. When a sufficient amount of light is obtained at the light outlet end of the light guide 31 with a single semiconductor laser, the power consumption can be reduced.

Further, since the semiconductor emitting element is better in light collection, the laser beam is hardly projected onto the surroundings of the light inlet end face of the light guide 31, whereby heat generation near the light inlet end of the light guide 31 is suppressed. Further, a light guide comprising a single optical fiber can be easily employed and the light guide can be reduced in its diameter.

An endoscope system in accordance with a second embodiment of the present invention will be described with reference to FIG. 2, hereinbelow. In the endoscope system 2 of this embodiment, the light source of the illumination means is disposed in the control portion. The elements analogous to those shown in FIG. 1 are given the same reference numerals and will not be described unless necessary.

The illumination means 40 comprises a light guide 41 which is positioned in the front end portion 14 of the insertion portion 10 at its front end and in the control portion 11 at its rear end, phosphors 32 positioned on the side of the light guide 31 near the front end thereof or in the front end portion 14 of the insertion portion 10, an illumination lens 33 provided in front of the phosphors 32, a semiconductor laser 42 which is positioned in the control portion 11 near the rear end of the light guide 31 and emits a laser beam which excites the phosphors 32, a collective lens 43 which collects the laser beam emitted from the semiconductor laser 42 on the light inlet end face of the light guide 31, a power source 36 which is provided in the processor 13 and supplies power to the semiconductor laser 42, and a power supply cable 37 connecting the power source 36 and the semiconductor laser 42.

The semiconductor laser 42 is a GaN series semiconductor laser and emits a laser beam (stimulating light) having a wavelength of near 405 nm (395 to 425 nm).

As in the first embodiment, a power source 36 is first turned on in response to operation of a manual switch not shown, and a laser beam of 405 nm is emitted from the semiconductor laser 42. The laser beam is collected by the collective lens 43 and enters the light guide 41. The laser beam propagated in the light guide 41 is emitted from the front end thereof which is positioned in the front end portion 14 of the insertion portion 10. The phosphors 32 are excited upon projection of the laser beam to emits white fluorescence light, a mixture of red, green, and blue fluorescence light. The white fluorescence light is projected onto an object part by way of the illumination lens 33 as illumination light.

In accordance with this embodiment, in addition to the effect inherent to the first embodiment, the universal cable 12 can be small in diameter since the semiconductor laser 42 is disposed in the control portion 11 and no light guide is disposed in the universal cable 12. Further, handling of the universal cable 12 is facilitated.

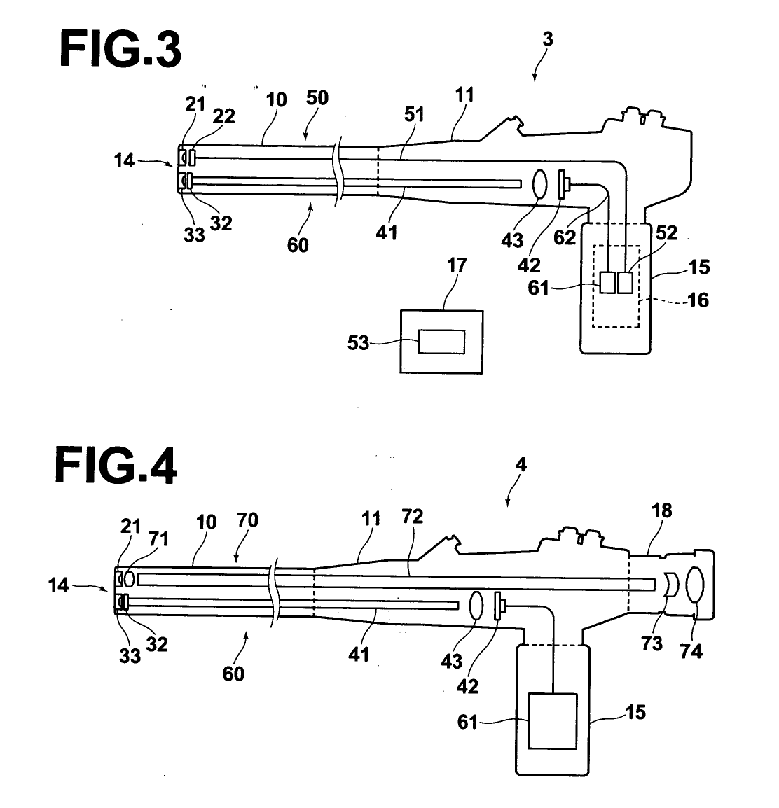

An endoscope system in accordance with a third embodiment of the present invention will be described with reference to FIG. 3, hereinbelow. The endoscope system 3 of this embodiment is in the form of a portable endoscope where no universal cable is necessary. The elements analogous to those shown in FIG. 1 are given the same reference numerals and will not be described unless necessary.

As shown in FIG. 3, the endoscope system 3 comprises an insertion portion 10 which is inserted into a body cavity or the like, a control portion 11 which is connected to a base end portion of the insertion portion 10, an extension 15 connected to the control portion 11 and a processor 17 remote from the endoscope body. A simplified processor 16 is provided in the extension 15.

The endoscope system 3 is further provided with an observing means 50 and an illumination means 60. The observing means 50 and the illumination means 60 are accommodated in the insertion portion 10, control portion 11 and the extension 15.

The observing means 50 comprises an objective lens 21 provided in the front end portion 14 of the insertion portion 10, a CCD 22 provided in an imaging position of the objective lens 21, a CCD cable 51 which is connected to the CCD 22 and extends to the insertion portion 10, control portion 11 and the extension 15, a signal transmitter 52 which is provided in the simplified processor 16 and is connected to the CCD cable 51, and a signal receiving/processing portion 53 which is provided in the processor 17 remote from the endoscope body. A monitor (not shown) is connected to the processor 17.

The illumination means 60 comprises a light guide 41 which is positioned in the front end portion 14 of the insertion portion 10 at its front end and in the control portion 11 at its rear end, phosphors 32 positioned on the side of the light guide 31 near the front end thereof or in the front end portion 14 of the insertion portion 10, an illumination lens 33 provided in front of the phosphors 32, a semiconductor laser 42 which is positioned in the control portion 11 near the rear end of the light guide 31, a collective lens 43, a power source 61 which is provided in the simplified processor 16 in the extension 15 and supplies power to the semiconductor laser 42, and a power supply cable 62 connecting the power source 61 and the semiconductor laser 42. Further, the simplified processor 16 effects a general control of the endoscope system including drive of a CCD 22 and the power source 61 for the semiconductor laser 42.

Operation of the endoscope system 3 in accordance with the third embodiment of the present invention described above will be described, hereinbelow. A power source 61 is first turned on in response to operation of a manual switch not shown, and a laser beam of 405 nm is emitted from the semiconductor laser 42. The laser beam is collected by the collective lens 43 and enters the light guide 41. The laser beam propagated in the light guide 41 is emitted from the front end thereof which is positioned in the front end portion 14 of the insertion portion 10. The phosphors 32 are excited upon projection of the laser beam to emits white fluorescence light, a mixture of red, green, and blue fluorescence light. The white fluorescence light is projected onto an object part by way of the illumination lens 33 as illumination light.

An image of the object part illuminated with the illumination light is formed on the image pick-up face of the CCD 22 by the objective lens 21. The CCD 22 takes the image of the object part and outputs an image signal representing the image of the object part to the CCD cable 51. The image signal is input into a signal transmitting portion 52 in the simplified processor 16 after propagating through the CCD cable 51. In the signal transmitting portion 52, the input image signal is transmitted by radio. The signal receiving/processing portion 53 provided in the processor 17 carries out predetermined processing to generate an image signal for an image display and the image signal for an image display is output to a monitor not shown. The observer observes an image of the object part displayed on the monitor.

In accordance with this embodiment, in addition to the effect inherent to the first embodiment, the universal cable can be unnecessary and the endoscope body can be small in size since the semiconductor laser 42 is disposed in the control portion 11 and the power source 61 is disposed in the extension 15. Further, a portable endoscope is realized by transmitting an image signal obtained by the CCD 22 by radio. It is preferred that the power source 61 comprises a disposal battery or a rechargeable battery. The simplified processor 16 may be provided in the control portion 11. In this case, the extension 15 may be in the form of an attachment in which other components such as an air reservoir for feeding air or a liquid reservoir for feeding liquid may be provided, whereby convenience of the endoscope system may be improved.

An endoscope system in accordance with a fourth embodiment of the present invention will be described with reference to FIG. 4, hereinbelow. In the endoscope system 4 of this embodiment, the object part is observed with the naked eye by the use of an image fiber without use of an image pick-up element. The elements analogous to those shown in FIG. 1 are given the same reference numerals and will not be described unless necessary.

As shown in FIG. 4, the endoscope system 4 comprises an insertion portion 10 which is inserted into a body cavity or the like, a control portion 11 which is connected to a base end portion of the insertion portion 10, an extension 15 connected to the control portion 11 and an eyepiece portion 18 connected to the control portion 11 opposite to the insertion portion 10.

The endoscope system 4 is further provided with an observing means 70 and an illumination means 60. The observing means 70 is accommodated in the insertion portion 10, control portion 11 and the eyepiece portion 18. While the illumination means 60 is accommodated in the insertion portion 10, control portion 11 and the extension 15.

The observing means 70 comprises an objective lens 21 provided in the front end portion 14 of the insertion portion 10, a lens 71 disposed behind the objective lens 21, an image fiber 72 which extends to the insertion portion 10, control portion 11 and the eyepiece portion 18, and eyepieces 73 and 74 which are disposed in the eyepiece portion 18.

An image of the object part illuminated with the illumination light is formed on an end face of the image fiber 72 by the objective lens 21 and the lens 71. The image fiber 72 is formed by a number of optical fibers bundled together and transmits the image to opposite end face thereof. The observer observes the image of the object part by way of the eyepieces 73 and 74.

In this embodiment, the CCD and the signal processing portion which processes an image signal output from the CCD become unnecessary and a portable endoscope system small in size can be realized. Further, the monitor becomes unnecessary, and accordingly, convenience of the endoscope system is improved.

Though, comprising a semiconductor laser emitting a laser beam near 405 nm in wavelength and phosphors 32 which emits white light (formed by red, green and blue light) upon stimulation by a laser beam of 405 nm in the embodiments described above, the illumination means need not be limited to such an arrangement. For example, as shown in FIG. 1, the illumination means may be an illuminations means 80 comprising a GaN series semiconductor laser which emits a laser beam having a wavelength of near 445 nm and phosphors 82 which scatter or transmit a laser beam having a wavelength of near 445 nm when a laser beam having a wavelength of near 445 nm is projected, and at the same time, emits red fluorescence light and green fluorescence light upon projection of blue light of about 445 nm. Blue light emitted from the semiconductor laser 81 is mixed with red light and green light emitted from the phosphors 82 to generate white illumination light. Further, phosphors which emit green light upon projection of blue light may be used, and a light source comprising a semiconductor laser emitting blue light and a semiconductor laser emitting red light may be used as the illumination means. In this case, red light may be used alone as the illumination light. Otherwise, phosphors emitting red light upon projection of green light may be used while a light source comprising a semiconductor laser emitting blue light and a semiconductor laser emitting green light is used.

Further, though a light source comprising a semiconductor laser is used in the embodiments described above, the light source need not be limited to those comprising a semiconductor laser. For example, the light source may comprise a laser diode. Further, a plurality of illumination means may be provided. In this case, the same illumination means may be provided in two, or the light source may be one and the guide line may be branched into a plural to provide phosphors on the front end of each branched guideline.

What is claimed is;:

1. An endoscope system having an insertion portion and a control portion connected to a base end portion of the insertion portion, wherein the improvement comprises
an illumination means comprising a light guide having a front end which is positioned near the front end of the insertion portion and a rear end which is positioned rearward of the base end of the insertion portion, phosphors disposed in the light guide toward the front end thereof and an illumination light source which is positioned toward the rear end of the light guide and emits stimulating light exciting the phosphors.

2. An endoscope system as defined in claim 1 in which the illumination light source is positioned in the control portion.

3. An endoscope system as defined in claim 1 in which the light source is a semiconductor light emitting element

4. An endoscope system as defined in claim 3 in which the light source is a semiconductor laser.

5. An endoscope system as defined in claim 1 in which the stimulating light is not shorter than 350 nm and not longer than 500 nm in wavelength.

6. An endoscope system as defined in claim 1 in which the illumination means emits white light, and the white light is fluorescence light emitted from the phosphors in response to projection of the stimulating light.

7. An endoscope system as defined in claim 1 in which the illumination means emits white light, and the white light is formed by the stimulating light and fluorescence light emitted from the phosphors in response to projection of the stimulating light.
